(19) United States
(12) Patent Application Publication
(10) Pub. No.: US 2026/0091205 A1
(43) Pub. Date: Apr. 2, 2026

(54) METHOD FOR ACCESSING A VESSEL USING A DUAL-ARTICULATING DEVICE

(71) Applicant: Baylis Medical Technologies Inc., Mississauga (CA)

(72) Inventors: Yara Alawneh, Mississauga (CA); Jodi Mackeil, Oakville (CA); Amanda Hartley, Caledon (CA); Ahmad Khayer Dastjerdi, Toronto (CA); Samantha Jane Lee, Mississauga (CA)

(21) Appl. No.: 19/335,723

(22) Filed: Sep. 22, 2025

Related U.S. Application Data
(60) Provisional application No. 63/701,089, filed on Sep. 30, 2024.

(51) Int. Cl. A61M 25/09; A61M 25/00

(52) U.S. Cl. CPC A61M 25/09041; A61M 2025/0008; A61M 2025/09075

(57) ABSTRACT

Systems and methods are disclosed for selectively accessing a target blood vessel and facilitating the advancement of treatment devices within the neurovasculature. The method includes causing an elongate device to adopt one or more curved configurations, which advance a distal end of the device into a target vessel, while avoiding adverse interactions with the vessel walls. The elongate device includes at least one deflection mechanism for deflection a portion of the shaft. Once the elongate device is located within a vessel, the curved configurations may be used to employ active stabilizing features to allow one or more inner devices to advance through the vessel in a controlled manner. The elongate device may then track over the inner device, using an inner device as a rail. An inner device can also be used to aim towards a target vessel.

### TECHNICAL FIELD

The disclosure relates to medical devices, and more particularly to methods and devices for accessing blood vessels within tortuous anatomy.

### BACKGROUND OF THE ART

Physicians typically use a combination of diagnostic and guiding catheters for neurological procedures. Diagnostic catheters are available in multiple shapes to maneuver through various shapes of vasculature. However, selecting an appropriate diagnostic shape prior to a procedure can be challenging due to varying patient anatomies. Certain anatomies pose more challenges than others, including type III aortic arches, bovine arches and tortuous vessels. The complexity of neurovascular anatomy can make accessing specific arteries difficult.

Unfavorable vascular anatomy can hinder the ability and speed of accessing the supra-aortic vessels. With age, the aortic arch tends to elongate, calcify, and lose compliance, leading to more angulated takeoffs of the great vessels. Consequently, standard catheters sometimes fail to catheterize the great vessels, as the diagnostic catheter often kicks back and herniates when the guidewire is advanced to achieve distal access. Another problem is that these arches frequently have atherosclerotic plaques on their walls and vessel ostia, increasing the risk of intraprocedural embolic complications during catheter manipulation and reformation.

A further clinical challenge in neurovascular procedures arises when physicians gain access to the origin of the target arteries. The goal is to navigate the distal end of the guiding catheter as close as possible to a treatment location, which is usually situated in the distal cranial arteries. The intricate curvature of the aortic arch, coupled with counteractive forces, impedes the desired advancement direction. Consequently, the axial load applied to the proximal end of the guiding catheter is not effectively transferred to the distal end. This disconnect poses a significant obstacle for physicians attempting to maneuver the catheter toward the distal cranial arteries, which can result in procedural delays or even failure.

It is therefore important that any device and associated techniques used for these procedures access target vessels quickly and not dislodge plaque buildup along vessel walls.

### BRIEF DESCRIPTION OF THE DRAWINGS

In order that the invention may be readily understood, embodiments of the invention are illustrated by way of examples in the accompanying drawings, in which:

FIG. 1 is an illustration of a catheter access system for facilitating the treatment of neurological procedures, in accordance with an embodiment of the present invention;

FIG. 2 is an illustration of a tri-axial system, in accordance with an embodiment of the present invention;

FIGS. 3A to 3C are illustrations of the articulating catheter distal end when inner devices are received within the articulating catheter, in accordance with another embodiment of the present invention;

FIGS. 4A and 4B are illustrations of an articulating catheter in accordance with an embodiment of the present invention;

FIGS. 4C and 4D are cross-sectional illustrations of the shaft of the articulating catheter in accordance with an embodiment of the present invention;

FIGS. 5A-5D are illustrations of various curved configurations of the articulating catheter in accordance with an embodiment of the present invention;

FIGS. 6A to 6C are illustrations of the articulating catheter in various curved configurations within a blood vessel in accordance with an embodiment of the present invention;

FIG. 7 is a flow diagram showing a method of selectively accessing a blood vessel as part of a procedure for treating a neurological condition in accordance with an embodiment of the present invention;

FIGS. 8A to 8H illustrate a method of selectively accessing a blood vessel within a neurovascular system in accordance with an embodiment of the present invention;

FIG. 9 is a flow diagram showing a method of selectively accessing a blood vessel as part of a procedure for treating a neurological condition in accordance with an alternative embodiment of the present invention; and

FIGS. 10A to 10F illustrate a method of selectively accessing a blood vessel within a neurovascular system in accordance with an alternative embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

Human neurovascular anatomy can be difficult to navigate because of its tortuous nature. There are numerous neurovascular procedures that require advancing treatment devices though this anatomy. Some examples of such procedures include the treatment of acute ischemic stroke, endovascular coil embolization, treating arteriovenous malformations (AVMs), and carotid angioplasty and stenting.

The systems and methods for facilitating advancement of treatment devices described herein may be used for, but are not limited to, any of the procedures described above. Some solutions described herein will be illustrated with respect to a mechanical thrombectomy procedure for treatment of acute ischemic stroke, but other applications are possible, and examples should not be considered limiting.

In one broad aspect, embodiments of the present invention comprise a method of selectively accessing a target vessel from an initial vessel using an elongate device, the method comprising the steps of: aligning a distal end of the elongate device with an opening of the target vessel; manipulating the elongate device to cause the elongate device to adopt a first curved configuration; manipulating the elongate device to cause the elongate device to adopt a second curved configuration; and advancing the distal end of the elongate device into the target vessel.

As a feature of this aspect, the elongate device includes at least two deflection regions, and the step of manipulating the elongate device to adopt the first curved configuration includes causing the elongate device to adopt a first curve in a first deflection region, and the step of manipulating the elongate device to adopt the second curved configuration includes causing the elongate device to adopt a second curve in a second deflection region.

As a feature of this aspect, the method further comprises the step of retracting an inner device longitudinally with respect to the elongate device such that a distal end of the inner device is proximal to the at least two deflection regions, the inner device being received within the elongate device.

As a feature of this aspect, the elongate device comprises at least one visual indicator, and the step of retracting the inner device comprising moving the inner device such that a distal of the inner device is proximal to the at least one visual indicator.

As a feature of this aspect, the method further comprises the steps of: advancing an inner device through the initial vessel towards the target vessel; and tracking the elongate device over the inner device.

As a feature of this aspect, the distal end of the elongate device is configured to form a smooth transition with an outer wall of the inner device when the inner device is positioned through the elongate device.

As a feature of this aspect, the method further comprises the step of: after the distal end of the elongate device has been advanced into the target vessel, advancing an inner device into the target vessel.

As a feature of this aspect, the method of further comprises the step of: advancing at least one treatment device through the elongate device.

As a feature of this aspect, the method further comprises a step of bracing at least one of the first deflection region and the second deflection region against a wall of the target vessel.

As a feature of this aspect, the initial vessel is an aortic arch and the target vessel is a brachiocephalic artery.

In another broad aspect, embodiments of the present invention comprise a method of selectively accessing a target vessel from an aortic arch using an articulating elongate device and a secondary elongate device, the secondary elongate device received within the articulating elongate device, the method comprising the steps of: positioning a distal end of the elongate device proximate to an opening of the target vessel; rotating the secondary elongate device about a longitudinal axis, whereby a distal end of the secondary elongate device aims towards the target vessel; manipulating the articulating elongate device to cause the elongate device to adopt a curved configuration; and advancing the distal end of the articulating elongate device into the target vessel.

As a feature of this aspect, the step of manipulating the elongate device to adopt the curved configuration comprises applying tension to at least one deflection mechanism associated with the articulating elongate device.

As a feature of this aspect, the articulating elongate device includes at least two deflection regions, and the step of manipulating the articulating elongate device to adopt the curved configuration includes causing the articulating elongate device to adopt a first curve in a first deflection region a second curve in a second deflection region.

As a feature of this aspect, the elongate device is operable in a relaxed configuration and in one or more curved configurations, and the steps of positioning a distal end of the articulating elongate device proximate to the opening of the target vessel occurs while the articulating elongate device is in the relaxed configuration.

As a feature of this aspect, the step of advancing the distal end of the articulating elongate device into the target vessel comprises tracking the articulating elongate device over the secondary elongate device.

As a feature of this aspect, the target vessel is a brachiocephalic artery.

As a feature of this aspect, the method further comprises the step of: advancing at least one treatment device through the articulating elongate device.

As a feature of this aspect, the method further comprises a step of bracing at least one of the first deflection region and the second deflection region against a wall of the target vessel.

As a feature of this aspect, the distal end of the articulating elongate device is configured to form a smooth transition with an outer wall of the secondary elongate device when the secondary elongate device is positioned through the articulating elongate device.

As a feature of this aspect, the method further comprises a step of locking the elongate device in at least one of the one or more curved configurations.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The inventors have developed a novel system and methods that can be used in the neurovasculature, specifically within the aortic arch and connecting vessels. The system can be used in complex anatomies such as type II or type III descended arches. The system can be used to advance devices from the aorta to the brachiocephalic artery, left common carotid artery or left subclavian artery.

The inventors have developed novel solutions to 1) advance through vasculature with reduced risk of dislodging plaque, 2) selectively access a targeted blood vessel and 3) add additional stabilizing features to reduce the risk of prolapse. Rounded features on device distal ends, and/or advancing by tracking will reduce risk of scraping plaque from vessel walls. The additional aiming and active bracing features are possible because a physician can manipulate the articulating catheter such that it can switch from a relaxed/flexible configuration to one or more biased configurations. In other words, it is operable between the relaxed and biased configuration. The biased configuration may also be referred to as a “curved”, “anchored”, or “braced” configuration.

The flexible configuration may be used when advancing the catheter within a blood vessel. The one or more biased configurations may be used to aim the distal end towards a targeted blood vessel, and to maintain the position of the catheter while one or more inner devices are moved therethrough and resist the tendency to prolapse. In other words, to actively brace the device in place within a blood vessel.

In some cases, plaque build-up may be present on vessel walls. In order to safely use a steerable device such as an articulating catheter, it must be advanced through the plaque lined vessels in such a way as to minimize or avoid dislodging plaque. The inventors have developed a novel vessel selection technique that is effective in situations where traditional methods for vessel access have failed. This technique allows for precise alignment of the articulating catheter tip with the vessel origin and enables the reforming of the articulating catheter inside the vessel by actuating the curves. This approach quickly gains access to the vessels with minimal manipulation, and minimal interaction with a vessel wall, thereby reducing the risk of plaque dislodgment.

### Catheter Access System

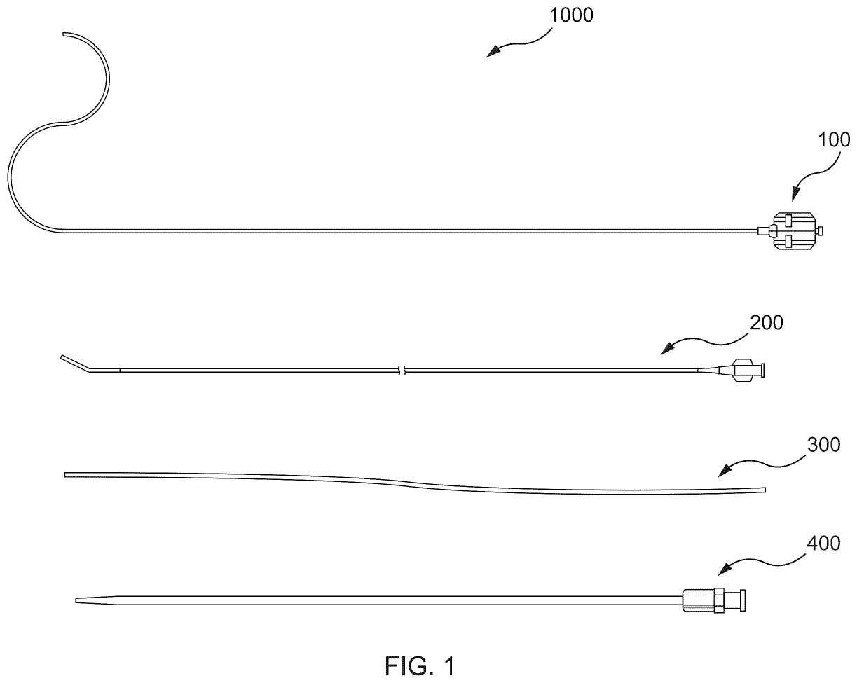

FIG. 1 shows a system which can be used in one or more neurovascular procedures described herein. In one embodiment, catheter access system 1000 includes a plurality of elongate/elongated medical devices including an articulating catheter 100, a secondary catheter 200, and a guidewire 300, together forming a “tri-axial system”. Articulating catheter 100 may also be referred to as “steerable catheter” or “anchoring catheter” and will be described in greater detail herein.

In some embodiments, catheter access system 1000 comprises a dilator (not shown) which can be used at the beginning of the procedure to facilitate delivering the components of the catheter access system 1000 into the patient.

Catheter access system 1000 is configured to be compatible with other treatment devices that can be used as part of an overall procedure to treat a neurological condition. Treatment devices include, but are not limited to, a stent retriever, an aspiration catheter, a microwire, a microcatheter, an intermediate catheter, a distal access catheter, other wires, other catheters, devices for delivering embolic agents, flow diverters, or other implantable devices.

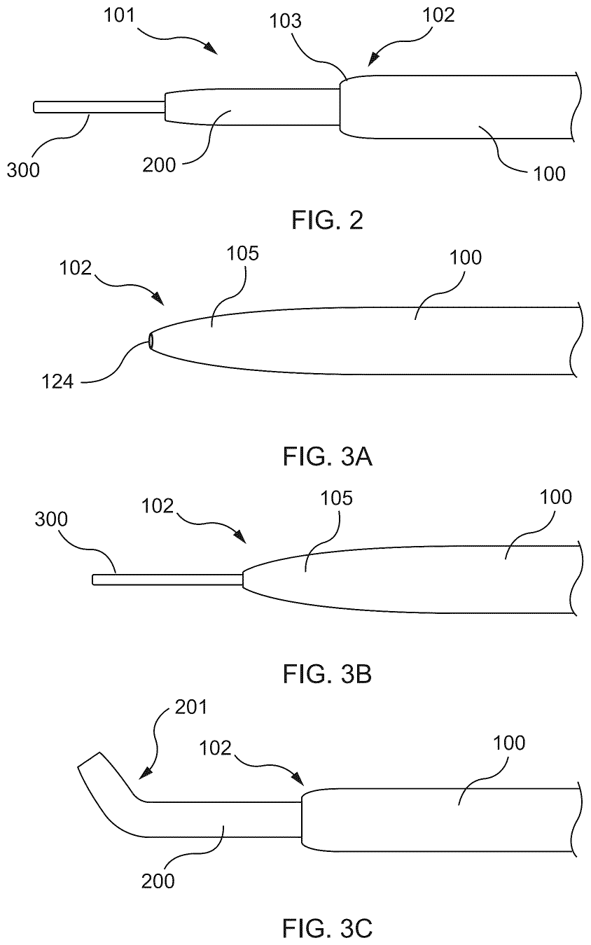

In one embodiment, secondary catheter 200 may be similar to commonly known flexible diagnostic catheters. In one embodiment, secondary catheter 200 has a curved distal end 201, as shown in FIG. 3C. (Also referred to as “pre-curved” or “fixed curve”) The curved distal end 201 may be used to aim toward a target vessel. In other embodiments, secondary catheter 200 has a straight distal end, as shown in FIG. 2.

In one embodiment, guidewire 300 may be any guidewire or wire guide, known by those skilled in the art, for example a flexible wire having a 0.035″ or 0.038″ outer diameter. Guidewire 300 may have a range of properties including various stiffness properties or distal shapes, for example straight, curved, angled. In some embodiments, guidewire 300 is an angled guidewire.

In one embodiment, articulating catheter 100 comprises a shaft including two or more deflection mechanisms, for example, one located at or near the distal end and one proximal to the distal end. These mechanisms facilitate forming multiple curve shapes and adapting to various diagnostic catheter shapes to aid vessel selection. In another embodiment, articulating catheter 100 comprises a single deflection mechanism along its shaft. In one example, a deflection mechanism includes a pull wire coupled to a pull mechanism, for example a pull ring, though other elements may be used as pull mechanisms, as will be described herein.

The deflection mechanisms (also referred to as “anchoring mechanisms”) are used to form different shapes and aim the distal end of the shaft and provide stability to facilitate advancement of the secondary catheter 200 through tortuous anatomy. In other words, the anchoring mechanisms provide stabilizing or “bracing” to maintain the position of the articulating catheter 100 throughout the procedure. Articulating catheter 100 is configured to track over secondary catheter 200 so it may be advanced through tortuous anatomy. In some embodiments, either or both articulating catheter 100 and secondary catheter 200 may comprise a hydrophilic coating to facilitate advancement through vessels. In some embodiments, a portion of either device comprises a hydrophilic coating and other portions of the device do not have a coating, for example, an uncoated portion at a curved region to facilitate stabilizing against a vessel wall.

With reference to FIG. 2, the articulating catheter 100, the secondary catheter 200, and the guidewire 300 can be arranged in a telescoping configuration and together form a tri-axial system 101. In one embodiment, distal end 102 of articulating catheter 100 comprises one or more features to create a smooth transition to secondary catheter 200, for example, distal end 102 comprises a taper 103. The smooth transition may be advantageous when advancing articulating catheter 100 through vessels walls and reduce the risk of scraping plaque off vessel walls. Additionally, the smooth transition reduces the interaction of the devices with the vessel walls, thereby decreasing patient risk. In another example, the distal end 102 may be rounded.

For clarification, smooth transition means zero or near zero gap between the inner diameter of the articulating catheter 100 primary lumen and the outer diameter of secondary catheter 200. The zero or near zero gap prevents articulating catheter 100 from scraping plaque from a vessel wall, if the catheter is moved along a vessel wall lined with plaque. In other words, the smooth transition reduces the “ledge effect”. Without something to block the gap of the distal opening 124, the articulating catheter 100 would act like a shovel and dislodge plaque. In one embodiment, the inner diameter of articulating catheter primary lumen 110 is between about 0.085″ and about 0.091″ and the outer diameter of the secondary catheter 200 is between about 0.080″ and about 0.085″.

In some embodiments, articulating catheter 100 can be steered or deflected while secondary catheter 200 extends out of distal end 102. In such an embodiment, the risk of distal end 102 scraping against a vessel wall and dislodging plaque is reduced because the distal opening of articulating catheter 100 is effectively blocked, and will not scrape against a vessel wall.

With reference to FIGS. 3A to 3C, in another embodiment, articulating catheter 100 includes an expandable portion 105 in the deflectable distal region 114 which comprises a material that is collapsible and expandible, such that it is preformed to have a small distal opening 124, which can be expanded when an inner device passes through it. In some embodiments, when the inner device is removed, the distal opening 124 returns to the small, preformed size and shape. When an inner device extends out of distal opening 124, the expandable portion 105 opens to the size of the inner device and forms a close contact and a smooth transition.

FIG. 3A shows articulating catheter 100 with no inner devices extending out of distal opening 124, which is in its smallest configuration, i.e., smallest distal opening 124. As shown in FIG. 3B, guidewire 300 extends out of distal opening 124, which is now slightly larger, and there is a smooth transition between the two devices. FIG. 3C shows secondary catheter 200 extending out of distal opening 124, which is even larger than the opening in FIG. 3B. The expandable portion 105 may allow an opening up to and including the size of articulating catheter 100 primary lumen inner diameter.

### Steerable Articulating Catheter

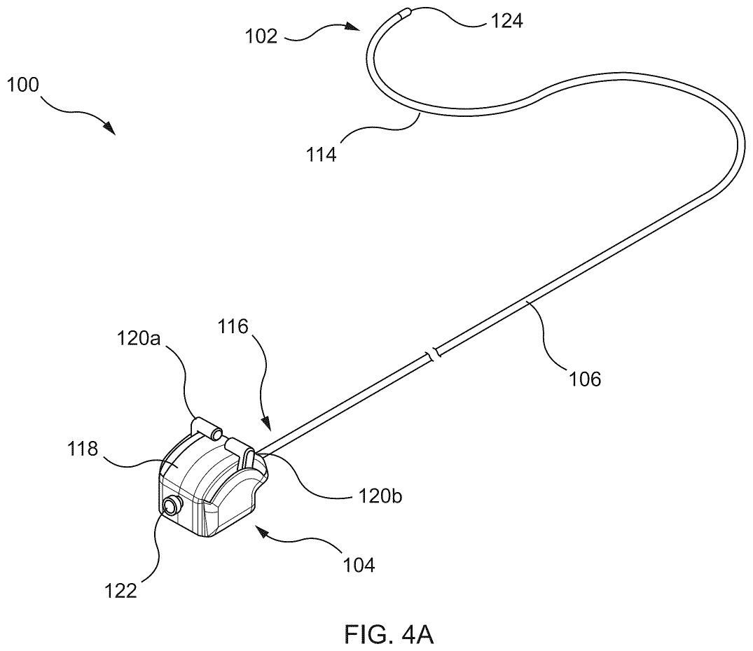

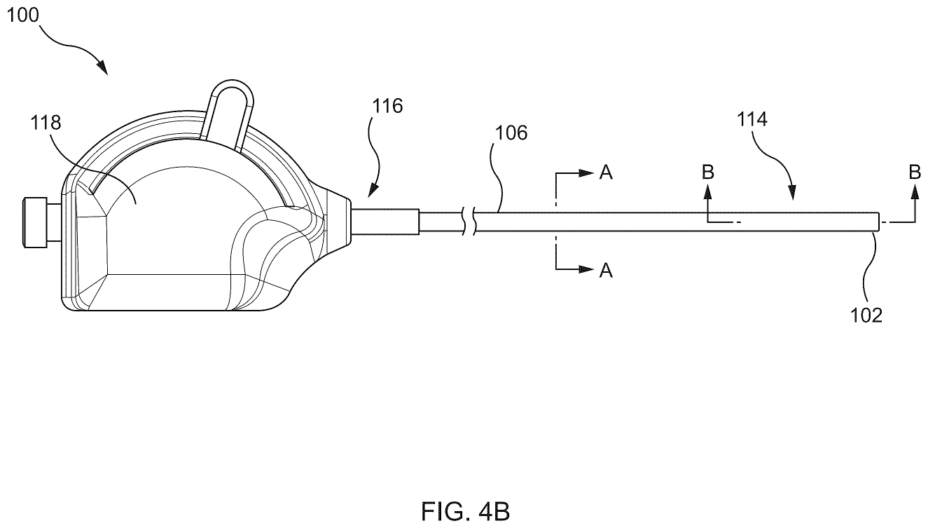

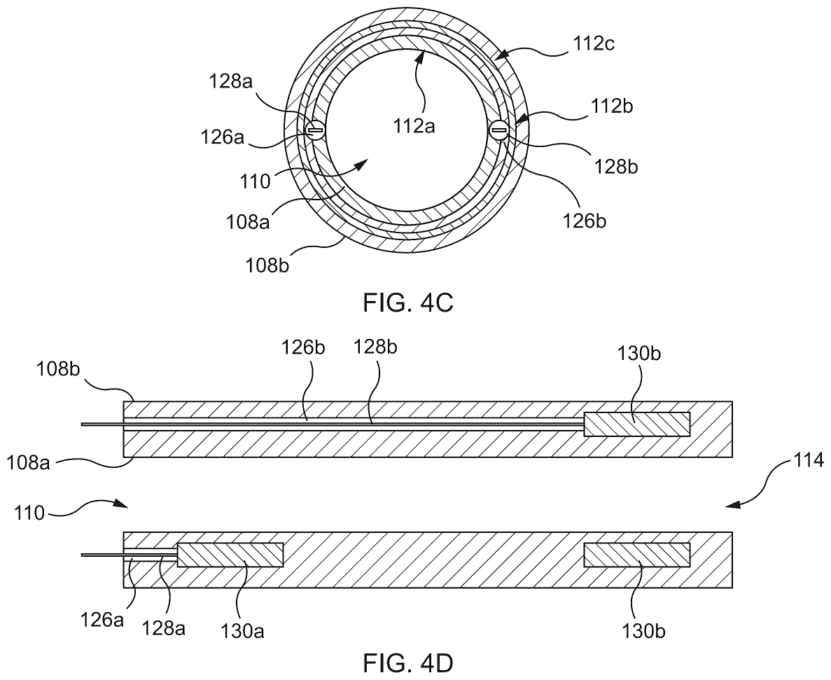

Referring to FIGS. 4A to 4D, according to one embodiment of the present invention, articulating catheter 100 has a distal end 102, a proximal end 104. Articulating catheter 100 includes a shaft 106 comprising inner wall 108a and outer wall 108b defining a primary lumen 110 extending between connection hub 122 and distal opening 124. Shaft 106 comprises a deflectable distal region 114 and a shaft proximal end 116.

In one embodiment, articulating catheter 100 comprises a control hub 118 located at proximal end 104. Shaft proximal end 116 is coupled to control hub 118. In one embodiment, control hub 118 comprises a connection hub 122 and one or more actuators 120, for example a first actuator 120a and a second actuator 120b. Actuator 120 may be a commonly known device such as a lever, knob, or a slider. Each actuator 120 is coupled to a deflection mechanism and can be manipulated to deflect a portion of shaft 106.

In some embodiments, connection hub 122 may be a luer connection, a hemostatic valve, or other known mechanism used to receive and create a fluid seal with an inner device, for example, secondary catheter 200, guidewire 300, or a treatment device. Each inner device may be inserted through connection hub 122, move longitudinally through primary lumen 110, and exit at distal opening 124. In other embodiments, a separate valve (not shown) is configured to connect to an opening on the control hub 118. In one example, the separate valve is a rotating hemostatic valve.

In another embodiment, not shown, control hub 118 comprises a handle. In some embodiments, shaft 106 comprises a plurality of regions of varying flexibility/rigidity extending along shaft 106.

FIG. 4C shows a cross section of shaft 106 taken along line A-A in FIG. 4B. In one embodiment, shaft 106 comprises one or more layers 112, for example a first layer 112a, second layer 112b, and third layer 112c. In one example, first layer 112a comprises a lubricious polytetrafluorethylene “PTFE” (or similar material), second layer 112b comprises a reinforced layer such as a braid, coil, or laser cut hypotube, and third layer 112c comprises a reflown thermoplastic layer. Shaft 106 also defines one or more wire lumens 126 located between inner wall 108a and outer wall 108b. In other embodiments, wire lumens 126 may be underneath, on top of, or within the second layer 112b. In other embodiments, shaft 106 has a lubricious layer, for example a hydrophilic or hydrophobic polymer (not shown).

In one embodiment, shaft 106 comprises two wire lumens 126 that are spaced substantially 180 degrees apart radially, as shown in FIG. 4C. In other embodiments, the radial distance between wire lumens 126a and 126b may be any other radial distance, for example offset by 90 degrees, 135 degrees, or any other distance to obtain various curved shapes. The wire lumens 126 may be positioned at any other point along the circumference.

Articulating catheter 100 comprises one or more pull wires 128 located within the one or more wire lumens 126. At the proximal end of the device, pull wires 128 are connected to one or more actuators 120 located in control hub 118. The distal end of each pull wire 128 is coupled to one or more pull mechanisms, for example pull rings 130, embedded within shaft 106. In one example, pull wires 128 are laser welded to pull rings 130.

In one embodiment, each actuator is coupled to one deflection mechanism, which includes a pull wire and a pull mechanism. Pull wire extends along the length of the shaft, and is coupled to the pull mechanism, which is embedded within the shaft, between inner and outer walls, or attached to the outside of the wall, or within the inner wall.

When actuated, tension is applied to pull wire 128 and shaft 106 deflects or curves in a region near the pull mechanism, creating a deflection region. In other words, a portion of the shaft adopts a curved configuration. The curved configuration changes the profile of shaft 106, and, when the deflection region is a distal region of the shaft, the device is “steered” or “aimed” within the vasculature. In other words, “aiming” means orienting or positioning the distal end so that the distal opening 124 faces a desired location, for example pointing at a target blood vessel, and any device that exits the distal opening 124 will be advanced towards that target location.

FIG. 4D shows a cross-section of a portion of shaft 106 taken along line B-B in FIG. 4B. In one embodiment, articulating catheter 100 comprises two pull wires 128a, 128b, coupled to pull rings 130a, 130b respectively. Pull ring 130a may be referred to as proximal pull ring and pull ring 130b may be referred to as distal pull ring. Pull wire 128a may be referred to as proximal pull wire and pull wire 128b may be referred to as distal pull wire. The portion of the shaft comprising pull ring 130a may be referred to as proximal anchor, and the portion of the shaft comprising pull ring 130b may be referred to as distal anchor. In one embodiment, proximal pull ring 130a is C-shaped and/or has a space or gap, to allow pull wire 128b to pass therethrough.

In some embodiments, the location of each anchor defines a deflection region along the shaft. In one example, a first deflection region is located proximal to proximal pull ring 130a, and a second deflection region is located between proximal pull ring 130a and distal to pull ring 130b. Such an embodiment may be referred to as a “dual-anchor catheter”. As used herein, anchors may be described as being “activated” or “actuated” when tension is applied to the anchor's corresponding pull wire. Similarly, “deactivating” an anchor means releasing tension from the anchor's pull wire.

Dual-anchor catheter is configured such that shaft 106 can adopt a double curve or “S-shaped” curve. S-shaped curve means a first curve in a first deflection region and second curve in a second deflection region, the second curve in a generally opposite direction to the first curve, as shown in FIG. 4A. In one embodiment, both curves share a common plane. In other embodiments, the first and second curves are in two different planes, for example, perpendicular to each other. In some embodiments, the radius of the first curve is different than the radius of the second curve.

The deflectable regions/curved regions may also be referred to as “anchor points”, and, in some embodiments, the rigidity at an anchor point increases when the shaft is curved in that region. The increased rigidity aids in bracing the device such that it supports advancement of an inner device therethrough. The double anchor catheter design can offer a high degree of articulation, which is critical for achieving a custom shape within the aortic arch, accommodating a wide range of anatomical variations among patients.

In one embodiment, a distal region of shaft 106 comprises a hydrophilic coating, except in regions near the curves, for example on the apex of each curve. When bracing against the vessel wall, the uncoated regions may facilitate bracing, in other words, resist slipping (longitudinal movement).

In one embodiment, each of actuators, 120a, 120b is configured to tension a single pull wire 128. In another embodiment, pull wires 128a and 128b are connected to a single actuator 120.

In further embodiments (not shown), articulating catheter 100 comprises two pull rings, two actuators, and four pull wires running through four wire lumens, where each pull ring is coupled to one actuator via two pull wires. In still further embodiments, articulating catheter 100 has additional pull rings to allow multiple curved portions of the shaft, for example three or more deflection regions allowing for three or more curves. In another embodiment (not shown), articulating catheter 100 is a unidirectional articulating catheter, and comprises one pull wire 128 connected to one pull ring 130.

In some embodiments, control hub 118 comprises one or more locking features (not shown), in order to lock the articulating catheter 100 in one or more curved configurations. Locking features may use any known mechanism, for example a switch, a push button, or a spring-loaded feature to engage locking teeth. Locking features may also comprise an auto-lock feature, where a push button must be depressed in order for shaft to deflect or return to the undeflected state. In one embodiment, actuator comprises a brake shoe that rotates about a pivot, the brake shoe is coupled to a lever that, when actuated, releases the brake shoe from the pivot.

In some embodiments, shaft 106 comprises visual indicators such as radiopaque features (not shown) that allow the portions of shaft 106 to be viewed under various known imaging modalities. Radiopaque features can include one or more marker bands, for example platinum iridium, located anywhere along shaft 106. In other embodiments, one or more layers 112 comprises a radiopaque filler that may provide visibility under fluoroscopy. In one example, the radiopaque filler is a high concentration of barium sulfate BaSO4.

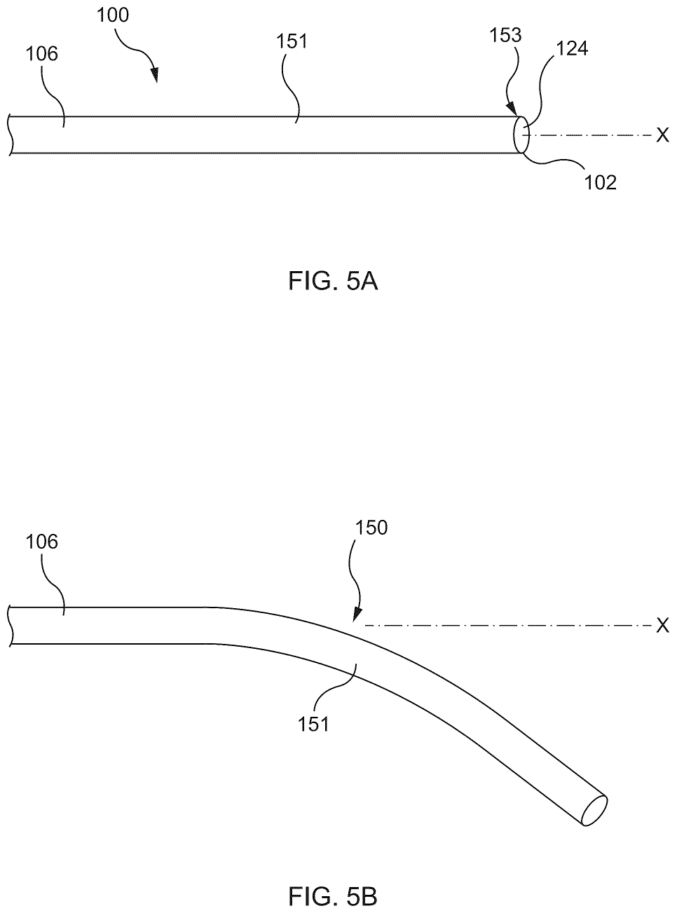

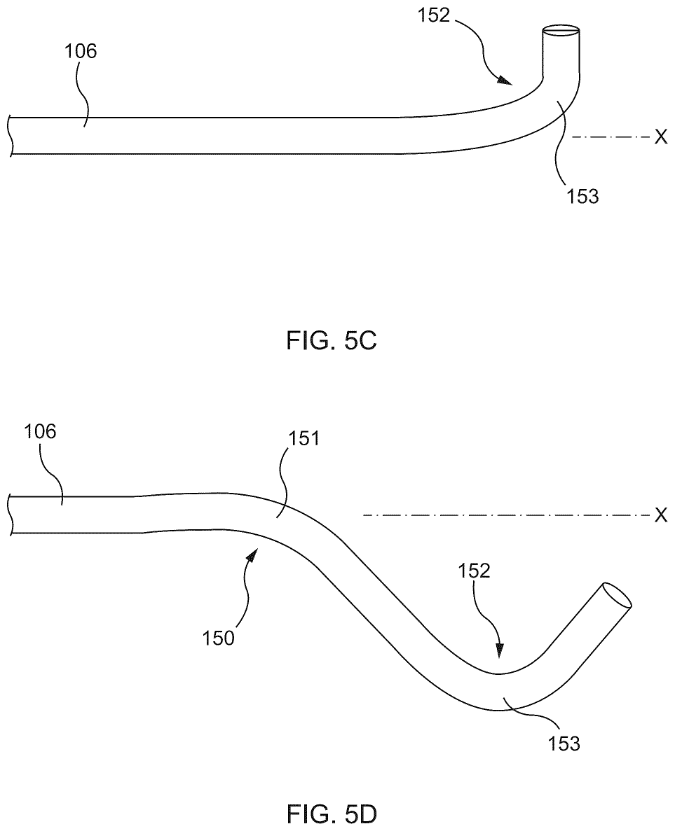

In one specific embodiment (not shown), shaft 106 comprises three marker bands: a first maker band near the distal end, a second marker band near the proximal pull ring 130a, and a third marker band proximal to the deflectable segments. In other words, the third marker band is proximal to proximal delectable region 151 and proximal curve 150 (see FIG. 5D). In such an embodiment, a physician may retract the distal end of an inner device, for example a secondary catheter, to a location proximal to the third marker band, prior to actuating the curves of the articulating catheter 100.

Referring to FIGS. 5A to 5D, articulating catheter 100 may toggle between a relaxed (flexible) configuration and at one or more biased (curved) configurations. In the relaxed configuration, tension is not applied to either deflection mechanism and the shaft is in its most flexible state, which may be advantageous for advancing through vasculature. In this state, it is possible for the shaft to curve at various regions, but it is not actively curved, that is, no tension is applied to either pull wire. In other words, at rest in the flexible configuration, shaft 106 is substantially straight, but deformable. Flexible configuration may also be referred to as a “slack configuration”.

FIG. 5A shows an illustration of shaft 106 in a flexible configuration, yet also substantially straight and in line with a shaft longitudinal axis X. While in a flexible configuration, shaft 106 can deflect relative to the X axis, however any curvature is not due to a tension in any pull wire, rather the curve is a result of the flexible properties of the shaft construction. In some embodiments, when shaft 106 is deformed, it will return to a substantially straight configuration upon release from a manual deflection. In other configurations, shaft 106 may retain a slight curve upon being released.

Referring to FIG. 5B, in a first biased configuration, tension is applied to the first (proximal) deflection mechanism, and not the second (distal) deflection mechanism. In other words, the proximal anchor is actuated. In such a configuration, shaft 106 adopts proximal curve 150 in the proximal deflection region 151.

Referring to FIG. 5C, in a second biased configuration, tension is applied to the second deflection mechanism, and not the first deflection mechanism. In other words, the distal anchor is actuated, such that shaft 106 adopts a distal curve 152 in the distal deflection region 153.

Referring to FIG. 5D, in a third biased configuration, tension is applied to both deflection mechanisms, such that shaft 106 adopts two curves in two deflection regions 151, 153, for example, proximal curve 150 and distal curve 152. Distal anchor and proximal anchor are both activated. This may also be referred to as a “double curve”, “S-curve”, or “S-shaped curved”.

One skilled in the art will appreciate that the number of degrees of curvature from the longitudinal X-axis can vary at one or both anchor points. A curve may be considered as any actuated deflection greater than zero degrees deviation from the longitudinal X-axis. In some examples, a curve may vary between 0 degrees and 180 degrees. In other examples, a curve may be greater than 180 degrees.

The deflection mechanisms described herein can be used to “aim” a distal end 102 towards a target blood vessel while a portion of shaft 106 is located within an initial vessel. “Aiming” means that the distal end 102 and distal opening 124 point towards the target vessel. For example, the initial vessel may be the aortic arch 10 and the target vessel may be the brachiocephalic artery 12. The brachiocephalic artery 12 branches off from the aorta, or, in other words, is in fluid communication with the aorta. The S-curve of the articulating catheter 100 facilitates advancement of one or more devices through the articulating catheter 100, into the brachiocephalic artery 12, as shown and described herein. Proximal curve 150 helps guide the device over the aortic arch 10 and distal curve 152 aims the distal end 102 towards brachiocephalic artery 12. While the brachiocephalic artery 12 is one example, the articulating catheter 100 can be manipulated to take other shapes to facilitate advancement into any number of other vessels, for example the left common carotid artery 18 or left subclavian artery 20.

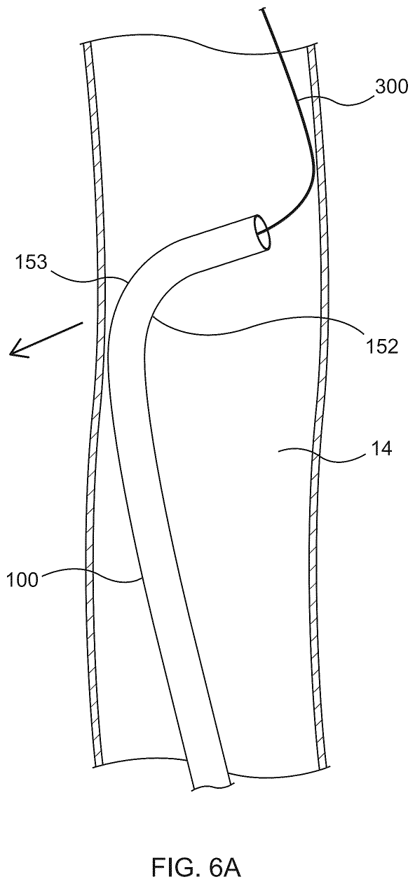

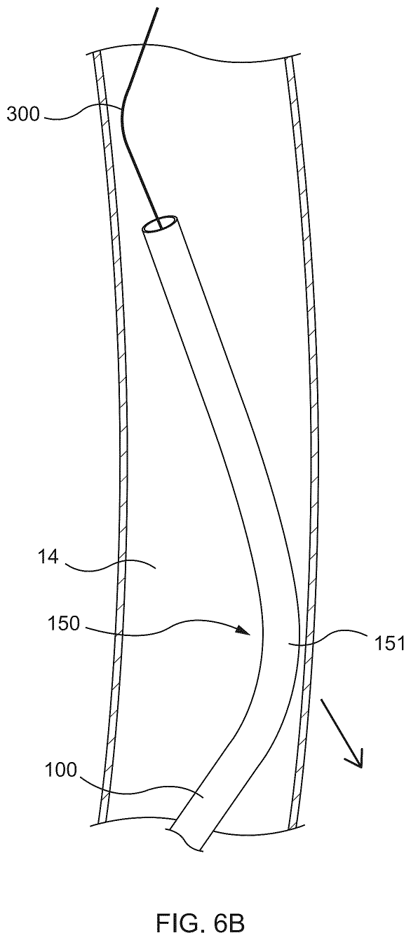

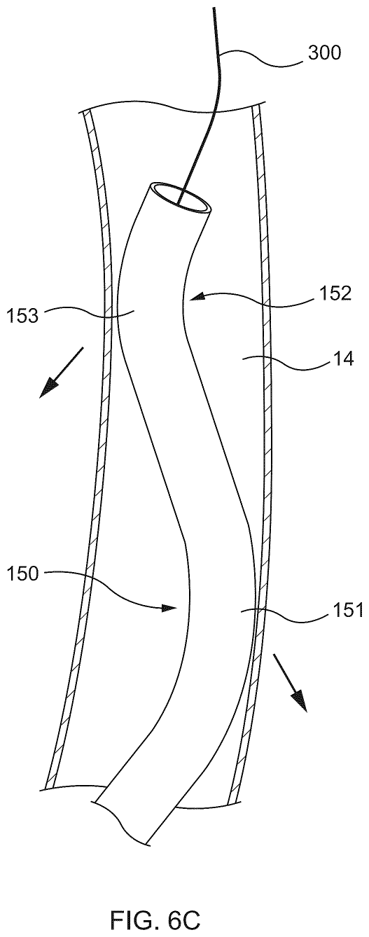

Referring to FIGS. 6A to 6C, when articulating catheter 100 is located within a blood vessel, actuating one or more deflection mechanisms will cause portions of the curved regions to come into contact with the vessel walls and facilitate bracing within the vessel. In other words, the contact with the vessel wall will resist longitudinal movement of the device within the vessel. In this way, the longitudinal position of the articulating catheter 100 is maintained while an inner device is advanced through primary lumen 110. In such a configuration, articulating catheter 100 is “actively” braced against a vessel wall.

As an inner device, for example, secondary catheter 200 or guidewire 300, is advanced through primary lumen 110, the inner device may exert a force that causes the articulating catheter 100 to move longitudinally.

FIG. 6A shows articulating catheter 100 having distal curve 152 where distal deflection region 153 contacts the wall of vessel, for example, right common carotid artery 14. As guidewire 300 is advanced out of distal opening 124, guidewire 300 exerts a force in the opposite direction to which it is travelling on portions of the articulating catheter 100. Some of the force that normally urges the articulating catheter 100 downward (in a proximal longitudinal direction) is absorbed by the vessel wall and helps to brace articulating catheter 100, limiting longitudinal movement. In other words, maintaining the position of the articulating catheter 100. The force is shown by the arrows in FIGS. 6A to 6C.

FIG. 6B shows articulating catheter 100 with proximal curve 150, and FIG. 6C shows both proximal curve 150 and distal curve 152. In FIG. 6C, deflection region 151 braces against the vessel wall at a first contact point and distal deflection region 153 braces against the vessel wall at a second contact point. In one example, the first and second contact points are radially offset by approximately 180 degrees.

In some embodiments, articulating catheter 100 comprises one or more features that assist bracing against a vessel wall. These features can include surface variations at locations that contact the vessel wall, for example a rough surface, or a series of bumps, grooves, or any other features that increases the friction between the vessel wall and the articulating catheter 100 outer wall 108b at a point of contact. In one specific example, the deflectable distal region 114 or the shaft 106 may comprise a hydrophilic coating, except at the apex of the curves 150, 152. Those portions of the shaft in the deflection regions 151, 153 include uncoated regions, i.e., no hydrophilic coating, such that there is in increased friction which facilitates bracing.

### Method of Accessing a Vessel (Multiple Deflection Mechanisms)

The inventors have developed a novel method to selectively access a blood vessel within tortuous anatomy quickly and safely, for example during the treatment of a neurological condition.

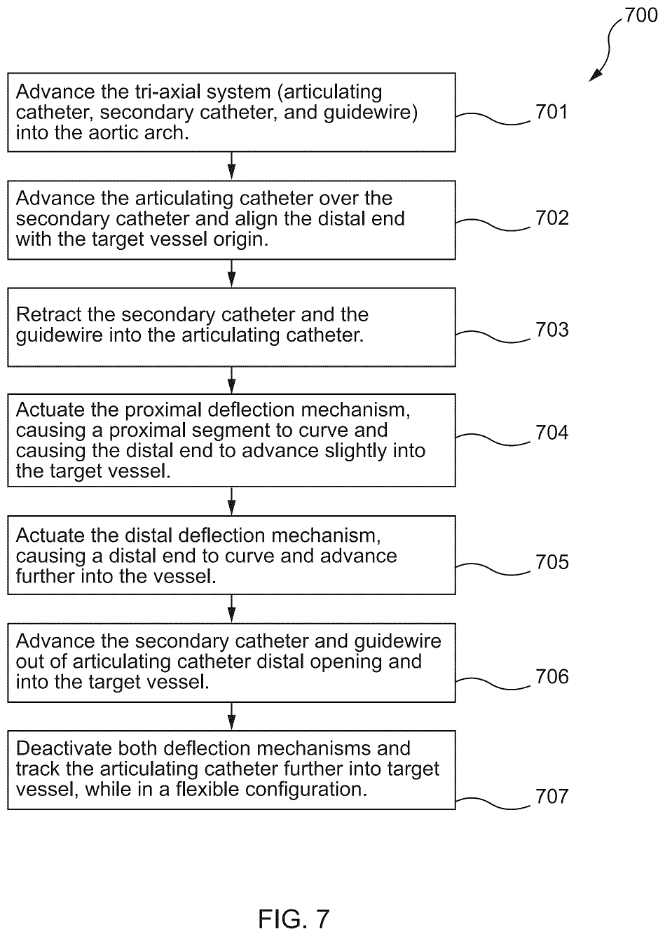

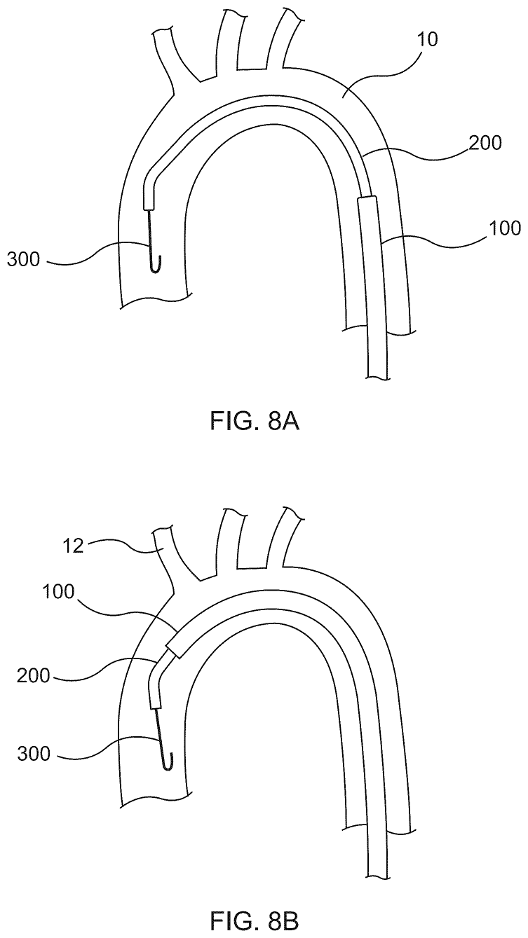

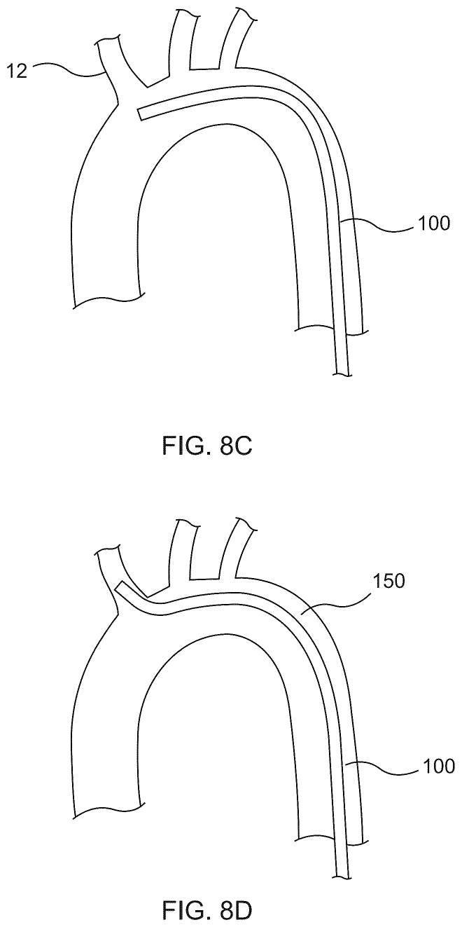

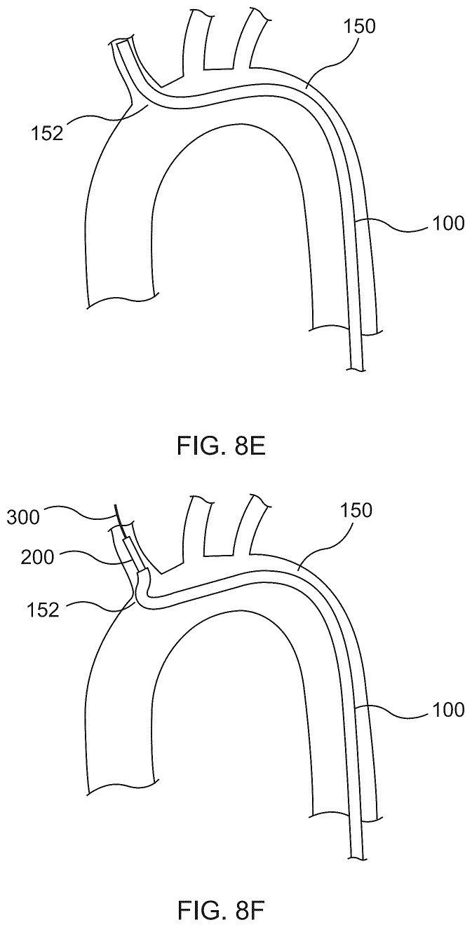

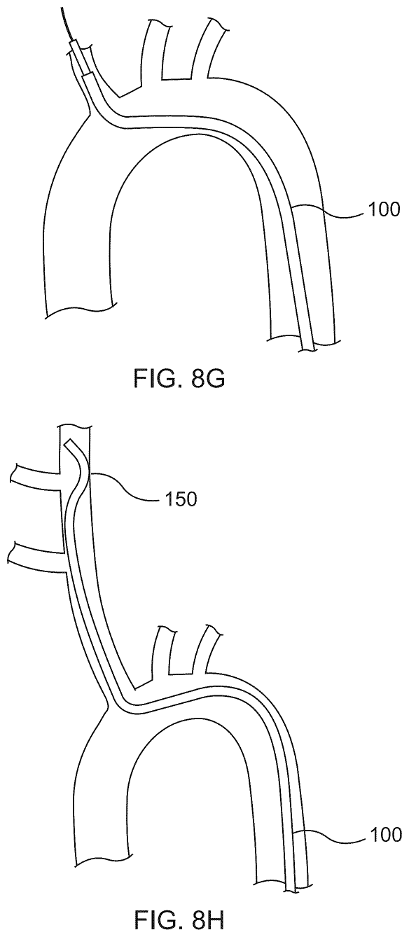

FIG. 7 illustrates a method for selectively accessing a target blood vessel using the devices described herein. The method comprises a series of planned steps designed to maximize safety and efficacy. Method 700 may be better understood with reference to FIGS. 8A to 8H. In this example, the anatomy has a Type III aortic arch and the target vessel is brachiocephalic artery 12, leading to right common carotid artery 14. Method 700 may be used to access other vessels as well, for example, the left common carotid artery 18 or the left subclavian artery 20.

Beginning at step 701, the tri-axial system 101, including the articulating catheter 100, the secondary catheter 200, and the guidewire 300, is advanced into the aortic arch 10, as shown in FIG. 8A. The guidewire 300 is inserted first, and the secondary catheter 200 tracks over the guidewire 300. Articulating catheter 100 is tracked over the secondary catheter 200.

At step 702, articulating catheter 100 is tracked over the secondary catheter 200 and distal end 102 aligned with the target vessel, in this example, the brachiocephalic artery 12. In one embodiment, the articulating catheter 100 is advanced in a flexible configuration. In some embodiments, distal end 102 is positioned approximately in the center, or as close as possible, to the center of the vessel origin, approximately mid-way between the vessel walls, as shown in FIG. 8B. In some examples, the guidewire 300 and secondary catheter 200 are advanced past the target vessel towards and/or into the ascending aorta.

In order to avoid interaction with vessel walls during steps 701 and 702, the articulating catheter 100 includes features designed to pass along vessel walls, without scraping plaque, if any is present. One way to avoid scraping plaque is to use the tri-axial system 101 with certain physical characteristics described herein, for example a tapered or rounded edge. In other words, articulating catheter 100 distal end 102 is configured to form a smooth transition with the outer wall of the secondary catheter 200, thereby reducing the risk of dislodging plaque.

At step 703, the secondary catheter 200 and guidewire 300 are retracted into the articulating catheter 100 primary lumen 110, and the articulating catheter 100 distal end 102 remains aligned with the origin of the target vessel, approximately at the mid-line of the vessel, as shown in FIG. 8C.

In some embodiments, guidewire 300 can be fully removed from articulating catheter 100 at this step. In some embodiments the secondary catheter 200 and/or guidewire 300 can be retracted such that the distal end(s) is/are located proximal to proximal deflection region 151, as observed by a physician using a visual indicator. In one embodiment, articulating catheter 100 has three marker bands: a first at the distal end, a second near the proximal pull ring, and a third proximal to proximal deflection region 151. In one example procedure, secondary catheter 200/guidewire 300 is/are retracted to a location proximal to the third marker band.

In other embodiments, the secondary catheter 200 is only slightly retracted, such that its distal end is substantially in line with, or slightly proximal to, distal end 102. In such an embodiment, secondary catheter 200 includes a flexible distal region, flexible enough to curve with articulating catheter 100.

In yet another embodiment, in some instances, secondary catheter 200 distal end may protrude from articulating catheter 100 distal end 102, while one or both deflection mechanisms are actuated.

At step 704, a physician actuates the proximal deflection mechanism, which causes the proximal deflection region 151 to deflect (downwards), such that the device adopts a first curved configuration, comprising proximal curve 150, as shown in FIG. 8D. In some cases, distal end 102 enters the target vessel.

At step 705, the physician actuates the distal deflection mechanism, which causes the distal deflection region 153 to curve and better align with and aim towards the target vessel, as shown in FIG. 8E. The physician can also advance the articulating catheter 100 longitudinally so that it advances further into the target vessel, either before, after, or during the distal anchor actuation.

At step 706, with the articulating catheter 100 in a desired location and in a biased configuration, the secondary catheter 200 and/or guidewire 300 can be advanced into the target vessel, as shown in FIG. 8F. The biased configuration helps to brace the articulating catheter 100 and resist movement as the inner devices are advanced through.

At step 707, once the secondary catheter 200 and/or guidewire 300 are advanced further into the target vessel, the proximal and distal anchors are deactivated such that articulating catheter 100 adopts a relaxed configuration, and then the articulating catheter 100 can be tracked over the secondary catheter 200/guidewire 300 further in the target vessel, as shown in FIG. 8G.

As an optional step, if advancing any of the inner devices is difficult, for example, if there is resistance or a tendency of one or more devices to slip while advancing, one or both anchors can be activated such that articulating catheter 100 takes biased configuration with a shape that braces against a vessel wall, as previously described herein, and shown in FIG. 8H. While in the braced configuration, one or both inner devices can be advanced while the secondary catheter facilitates bracing, i.e., resists longitudinal movement in the direction opposite of the advancing direction.

The articulating catheter 100 may be advanced in a relaxed configuration, using an inner device as a rail. After the articulating catheter 100 is advanced a certain distance, the articulating catheter 100 is put into a braced configuration and one or both inner devices are advanced while the articulating catheter 100 is braced against a vessel wall. The articulating catheter 100 can then adopt a flexible configuration and track again over an inner device. A physician may activate or deactivate the anchors as needed in any number of combinations, to switch between a relaxed configuration and a curved configuration, thereby facilitating advancement of the articulating catheter 100 through tortuous anatomy.

In this way, in addition to aiming, the anchoring features on the articulating catheter 100 may be used to provide active bracing for advancement of inner devices during a neurological procedure. Once the articulating catheter 100 is advanced far enough into the target vessels and close to a treatment location, the guidewire 300 and secondary catheter 200 can be removed and the neurological treatment procedure can continue, i.e., treatment devices can be delivered to the target location.

In an alternative embodiment of the present invention, the method may be used with an articulating catheter that has only one deflection mechanism, i.e., one pull ring for deflecting one section of the shaft, for example a distal section. For such an embodiment, steps 701 to 707 are the same, except there is no step 705. Only a single deflection mechanism is actuated at step 704.

### Alternative Method of Accessing a Vessel

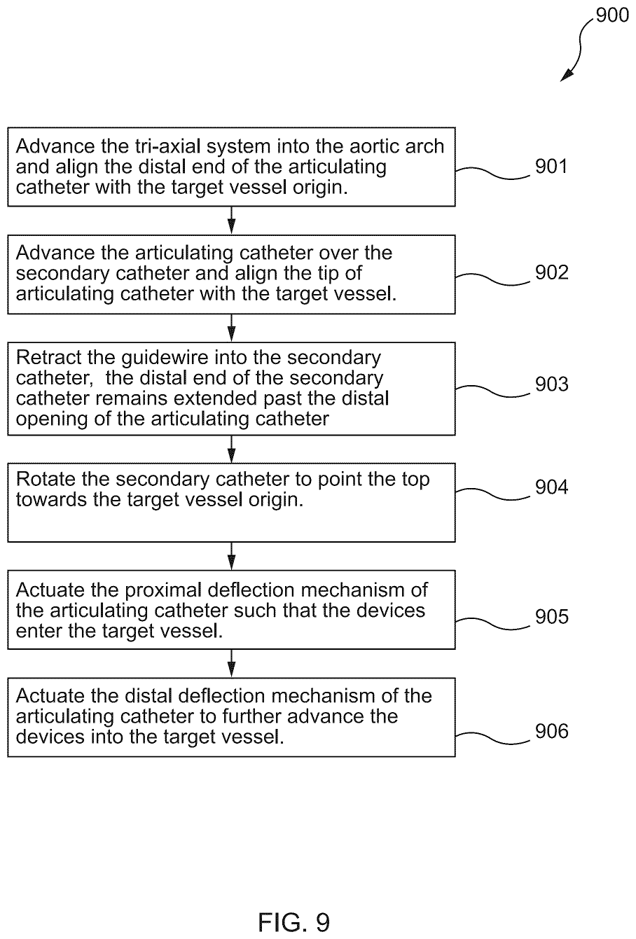

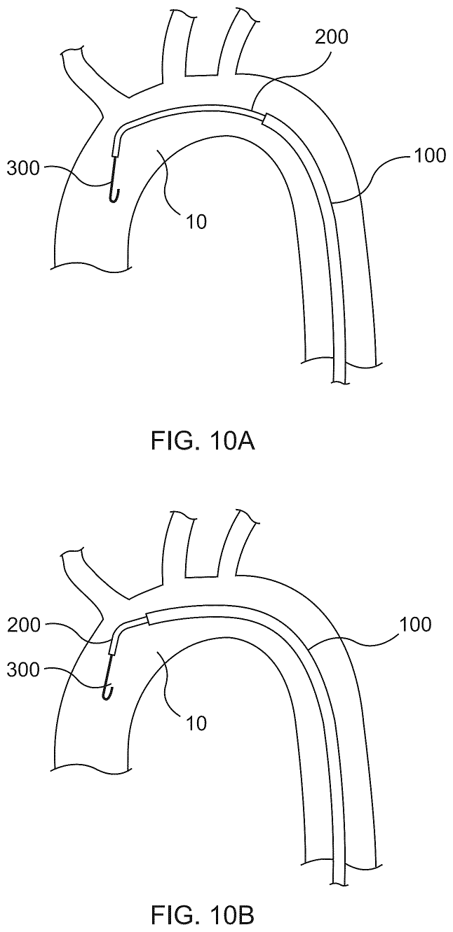

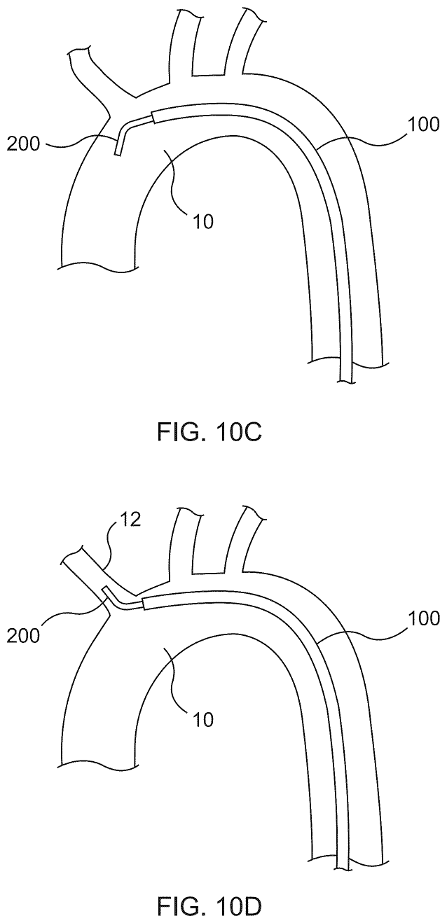

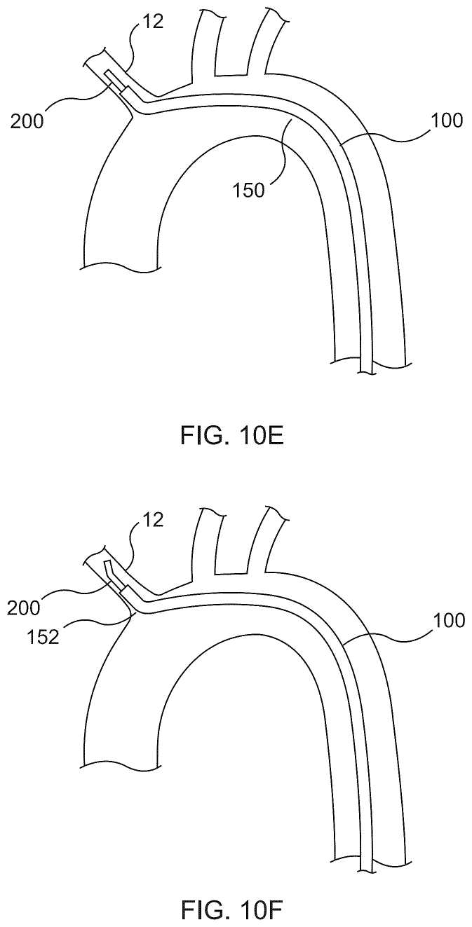

FIG. 9 illustrates an alternative method for selectively accessing a target blood vessel using the devices described herein. Method 900 may be better understood with reference to FIGS. 10A to 10F. Similar to method 700, the anatomy has a Type III aortic arch and the target vessel is brachiocephalic artery 12, leading to right common carotid artery 14. Method 900 repeats several steps of method 700 described herein.

Beginning at step 901, the tri-axial system 101, including articulating catheter 100, secondary catheter 200, and guidewire 300, is advanced into the aortic arch 10. In some examples, guidewire 300 is inserted first, secondary catheter 200 tracks over the guidewire 300, and articulating catheter 100 tracks over secondary catheter 200. In one embodiment, secondary catheter 200 comprises a pre-curved distal end. The curved distal end is flexible enough to straighten and advance through primary lumen 110 and then adopt a curved shape upon existing distal opening 124.

At step 902, the articulating catheter 100 distal end 102 is positioned at a desired location, close to the target vessel, in this example. The distal end 102 is positioned slightly short of the vessel origin (to the right in the figure), as shown in FIG. 10B.

At step 903, guidewire 300 is retracted into the secondary catheter 200. The distal end of secondary catheter 200, including the curve, remains extended out of articulating catheter 100 distal end 102, as shown in FIG. 10C.

At step 904, secondary catheter 200 can be rotated or “torqued” such that the distal end and curve enters the target vessel, as shown in FIG. 10D. For clarification, rotating or “torquing” in this case, means rotating the secondary catheter 200 about a longitudinal axis of the catheter, in line with the primary lumen. In some examples, the curved distal end will aim towards the target vessel and in other examples, the curved distal end enters the target vessel, depending on anatomy and the positioning of each device. A physician can also move one or both devices longitudinally to gain access to the target vessel.

At step 905, the physician actuates the proximal deflection mechanism, which causes the articulating catheter 100 and secondary catheter 200 to gain further access into the target vessel, as shown in FIG. 10E. Articulating catheter 100 adopts proximal curve 150.

At step 906, the physician actuates the distal deflection mechanism, which causes the articulating catheter 100 and secondary catheter 200 to gain even further access into the target vessel, as shown in FIG. 10F. Articulating catheter 100 adopts distal curve 152.

At step 905 and/or step 906, the physician can also advance the articulating catheter 100 longitudinally so that it advances further into the target vessel, either before, after, or during the either deflection mechanism actuation.

Optional bracing steps described herein can be used as needed to advance one or more devices through the vessels.

In one example, the procedure may be the treatment of acute ischemic stroke, and the treatment location may be a location having a clot, such as the M1-sphenoidal branch or M2-insular branch of the middle cerebral artery, anywhere in the middle cerebral, or anywhere in the internal carotid artery. A treatment device such as a stent retriever or aspiration catheter may be advanced through the articulating catheter to the treatment location.

The devices and methods disclosed herein can be used for any number of applications that require delivering devices to the neurovascular system, including mechanical thrombectomy, stenting, coiling, pipeline/flow diversion, stent-assisted coiling, aneurysm embolization, and balloon assisted coiling.

The devices and methods disclosed herein may be used in other areas of a patient's body, including in renal artery selection, for example, the devices and methods can be used to access arteries that branch from the abdominal aorta, including left and right renal arteries, celiac trunk, superior mesenteric artery, and inferior mesenteric artery. Another application may be for lead placement in the heart where stabilizing a lead in a complex cardiac environment is difficult.

The devices and methods disclosed herein offer advantages over existing articulating catheters, where the tip can end up pointed into the vessel wall, which can be a risk for dissection of the vessel wall from contrast injections or advancement of treatment devices through the articulating catheter. With traditional articulating catheters there is nothing the user can do to change which way the tip is pointed so they often have to pull it back until it points in a safer direction. With the device described herein, the tip position can be modified by articulating the curves. This feature would allow the physicians to keep the articulating catheter in a more distal location regardless of how the tip naturally lands.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention.

1. A method of selectively accessing a target vessel from an initial vessel using an elongate device, the method comprising the steps of:
aligning a distal end of the elongate device with an opening of the target vessel;
manipulating the elongate device to cause the elongate device to adopt a first curved configuration;
manipulating the elongate device to cause the elongate device to adopt a second curved configuration; and
advancing the distal end of the elongate device into the target vessel.

2. The method of claim 1, wherein the elongate device includes at least two deflection regions, and wherein the step of manipulating the elongate device to adopt the first curved configuration includes causing the elongate device to adopt a first curve in a first deflection region, and the step of manipulating the elongate device to adopt the second curved configuration includes causing the elongate device to adopt a second curve in a second deflection region.

3. The method of claim 2, wherein the method further comprises the step of retracting an inner device longitudinally with respect to the elongate device such that a distal end of the inner device is proximal to the at least two deflection regions, the inner device being received within the elongate device.

4. The method of claim 3, wherein the elongate device comprises at least one visual indicator, and the step of retracting the inner device comprising moving the inner device such that a distal of the inner device is proximal to the at least one visual indicator.

5. The method of claim 1, further comprising the steps of:
advancing an inner device through the initial vessel towards the target vessel; and
tracking the elongate device over the inner device.

6. The method of claim 5, wherein the distal end of the elongate device is configured to form a smooth transition with an outer wall of the inner device when the inner device is positioned through the elongate device.

7. The method of claim 1, further comprising the step of:
after the distal end of the elongate device has been advanced into the target vessel, advancing an inner device into the target vessel.

8. The method of claim 1, further comprising the step of:
advancing at least one treatment device through the elongate device.

9. The method of claim 2 further comprising a step of bracing at least one of the first deflection region and the second deflection region against a wall of the target vessel.

10. The method of claim 1, wherein the initial vessel is an aortic arch and the target vessel is a brachiocephalic artery.

11. A method of selectively accessing a target vessel from an aortic arch using an articulating elongate device and a secondary elongate device, the secondary elongate device received within the articulating elongate device, the method comprising the steps of:
positioning a distal end of the elongate device proximate to an opening of the target vessel;
rotating the secondary elongate device about a longitudinal axis, whereby a distal end of the secondary elongate device aims towards the target vessel;
manipulating the articulating elongate device to cause the elongate device to adopt a curved configuration; and
advancing the distal end of the articulating elongate device into the target vessel.

12. The method of claim 11, wherein the step of manipulating the elongate device to adopt the curved configuration comprises applying tension to at least one deflection mechanism associated with the articulating elongate device.

13. The method of claim 11, wherein the articulating elongate device includes at least two deflection regions, and wherein the step of manipulating the articulating elongate device to adopt the curved configuration includes causing the articulating elongate device to adopt a first curve in a first deflection region a second curve in a second deflection region.

14. The method of claim 11, wherein the elongate device is operable in a relaxed configuration and in one or more curved configurations, and wherein the steps of positioning a distal end of the articulating elongate device proximate to the opening of the target vessel occurs while the articulating elongate device is in the relaxed configuration.

15. The method of claim 11, wherein the step of advancing the distal end of the articulating elongate device into the target vessel comprises tracking the articulating elongate device over the secondary elongate device.

16. The method of claim 11, wherein the target vessel is a brachiocephalic artery.

17. The method of claim 11, further comprising the step of:
advancing at least one treatment device through the articulating elongate device.

18. The method of claim 13 further comprising a step of bracing at least one of the first deflection region and the second deflection region against a wall of the target vessel.

19. The method of claim 11, wherein the distal end of the articulating elongate device is configured to form a smooth transition with an outer wall of the secondary elongate device when the secondary elongate device is positioned through the articulating elongate device.

20. The method of claim 14 further comprising a step of locking the elongate device in at least one of the one or more curved configurations.
